(19) United States
(12) Patent Application Publication
(10) Pub. No.: US 2026/0108187 A1
(43) Pub. Date: Apr. 23, 2026

(54) DIFFUSE REFLECTANCE SPECTROSCOPIC MEASUREMENT SYSTEM FOR A MEDIUM TO BE ANALYSED

(71) Applicant: COMMISSARIAT A L'ÉNERGIE ATOMIQUE ET AUX ENERGIES ALTERNATIVES, Paris (FR)

(72) Inventors: Benoit RACINE, GRENOBLE CEDEX 09 (FR); Aurélien SUHM, GRENOBLE CEDEX 09 (FR); Christelle LAUGIER, GRENOBLE CEDEX 09 (FR); Angélique RASCLE, GRENOBLE CEDEX 09 (FR); Luc ANDRE, GRENOBLE CEDEX 09 (FR)

(73) Assignee: COMMISSARIAT A L'ÉNERGIE ATOMIQUE ET AUX ENERGIES ALTERNATIVES, Paris (FR)

(21) Appl. No.: 19/356,511

(22) Filed: Oct. 13, 2025

(30) Foreign Application Priority Data
Oct. 14, 2024 (FR) FR2411097

(51) Int. Cl. A61B 5/1455; A61B 5/00; A61B 5/145

(52) U.S. Cl. CPC A61B 5/1455; A61B 5/0075; A61B 5/14532; A61B 5/4875; A61B 2562/0238; A61B 2562/04; A61B 2562/12

(57) ABSTRACT

A diffuse reflectance spectroscopic measurement system for a medium to be analysed, including a substrate; an electroluminescent device, arranged on a first zone of a surface of the substrate, and configured to emit light radiation, the electroluminescent device having an output surface, through which the light radiation is transmitted towards the medium to be analysed; a first photodetector, arranged on a portion of the output surface of the electroluminescent device so as to receive a portion of the light radiation and so that the output surface retains a free zone; a second photodetector, arranged on a second zone of the surface of the substrate so as to receive the light radiation transmitted by the free zone and diffusely reflected by the medium to be analysed.

### TECHNICAL FIELD

The invention relates to the technical field of diffuse reflectance spectroscopy.

The invention is notably applicable in the non-invasive identification of biochemical components (for example, oxygenation level, blood sugar level, hydration level, etc.) in biological tissue such as skin.

### PRIOR ART

A diffuse reflectance spectroscopic measurement system known in the prior art, notably from document WO 2023/094887 A1, comprises:
a substrate, comprising a surface intended to be oriented towards a medium to be analysed, the surface having first and second adjacent zones;
a light source, arranged on the first zone of the surface of the substrate, and configured to emit light radiation in a wavelength range;
an output coupler, arranged on the second zone of the surface of the substrate, and having an output surface, through which a majority portion (for example, 99%) of the light radiation is transmitted towards a medium to be analysed;
a wavemeter, arranged on the second zone of the surface of the substrate;
a photodetector, arranged on the second zone of the surface of the substrate so as to receive the light radiation transmitted by the output surface of the output coupler and diffusely reflected by the medium to be analysed.

The light radiation emitted by the light source is split so that:
(i) a majority portion (for example, 99%) of the emitted light radiation is guided towards the output coupler in order to transmit the majority portion of the light radiation to the medium to be analysed;
(ii) a minority portion (for example, 1%) of the emitted light radiation is guided towards the wavemeter in order to deduce the light power emitted by the light source.

Determining the number of photons emitted by the light source towards the medium to be analysed is essential for obtaining substantive diffuse reflectance spectroscopy.

Such a diffuse reflectance spectroscopic measurement system of the prior art is not entirely satisfactory in that the presence of waveguides (towards the output coupler and towards the wavemeter) makes manufacturing the measurement system more complex and introduces significant optical losses, thereby reducing the efficiency of the system (increased energy consumption). Furthermore, the output coupler and the wavemeter occupy a substantial area of the second zone of the surface of the substrate, introducing a certain amount of lateral bulk that can cause discomfort when the measurement system is carried by a patient.

### DISCLOSURE OF THE INVENTION

The aim of the invention is to overcome all or some of the aforementioned disadvantages. To this end, the aim of the invention is a diffuse reflectance spectroscopic measurement system for a medium to be analysed, comprising:
a substrate, comprising a surface intended to be oriented towards the medium to be analysed, the surface having first and second adjacent zones;
an electroluminescent device, arranged on the first zone of the surface of the substrate, and configured to emit light radiation in a wavelength range, the electroluminescent device having an output surface, through which the light radiation is transmitted towards the medium to be analysed;
a first photodetector, called reference photodetector, arranged on a portion of the output surface of the electroluminescent device so as to receive a portion of the light radiation and so that the output surface retains a free zone;
a second photodetector, called measurement photodetector, arranged on the second zone of the surface of the substrate so as to receive the light radiation transmitted by the free zone of the output surface of the electroluminescent device and diffusely reflected by the medium to be analysed.

Thus, such a system according to the invention dispenses with the presence of waveguides compared to the prior art. Indeed, on the one hand, there is no output coupler since the light radiation is transmitted directly via the output surface of the electroluminescent device. On the other hand, the reference photodetector (used to deduce the light power emitted by the electroluminescent device) is arranged on a portion of the output surface of the electroluminescent device. In addition, stacking the reference photodetector on the electroluminescent device results in reduced lateral bulk compared to the prior art.

The system according to the invention can comprise one or more of the following features.

According to one feature of the invention, the electroluminescent device comprises at least one organic light-emitting diode.

Thus, one advantage provided by such an electroluminescent device is that it achieves a wide emission angle with limited loss of luminance between the centre and the periphery, such that the output surface is homogeneous.

According to one feature of the invention, the first photodetector comprises at least one organic photodiode.

Thus, one advantage provided by such a photodetector is that it can be easily manufactured on the electroluminescent device, especially when the electroluminescent device is organic.

According to one feature of the invention, the second photodetector comprises at least one organic photodiode.

Thus, one resulting advantage is that the first and second photodetectors can be easily manufactured concomitantly when they are organic.

According to one feature of the invention, the electroluminescent device successively comprises:
a first electrode, arranged on the first zone of the surface of the substrate;
a stack of layers, comprising an emissive layer configured to emit the light radiation in the wavelength range;
a second electrode, transparent in the wavelength range, and having a surface defining the output surface of the electroluminescent device.

The second electrode must be transparent in the wavelength range in order to transmit the light radiation emitted by the emissive layer. The first electrode is advantageously reflective in the wavelength range in order to reduce optical losses. Thus, a constraint is removed from the nature of the substrate, which may not be reflective in the wavelength range in order to reduce optical losses. However, if the first electrode is not reflective in the wavelength range, a substrate then can be used that is reflective in the wavelength range in order to reduce optical losses.

According to one feature of the invention, the first photodetector successively comprises:
a first electrode, transparent in the wavelength range, and arranged on a portion of the output surface of the electroluminescent device;
a stack of layers, comprising an absorbent layer configured to absorb the light radiation in the wavelength range;
a second electrode, which is reflective in the wavelength range.

The first electrode is transparent in the wavelength range so that the light radiation emitted by the electroluminescent device can reach the absorbent layer. The second electrode is reflective in the wavelength range so that the residual light radiation, not absorbed by the absorbent layer, does not propagate into the medium to be analysed.

According to one feature of the invention, the first electrode of the first photodetector is arranged on a lateral portion of the output surface.

Thus, one resulting advantage involves facilitating access for electrically connecting the first photodetector, while optimising the distribution of light radiation towards the medium to be analysed.

According to one feature of the invention:
the output surface has an area, denoted “A_{S}”;
the first electrode of the first photodetector occupies an area, denoted “A_{P}”, satisfying:

$${A}_{P} = \frac{{A}_{S}}{n}$$

where “n” is a natural number that is greater than or equal to 2.

Thus, one resulting advantage involves facilitating the determination of the number of photons emitted by the electroluminescent device towards the medium to be analysed in order to obtain substantive diffuse reflectance spectroscopy. The total number of photons emitted at a given instant by the electroluminescent device corresponds to “n” times the number of photons detected by the first photodetector at the given instant. The number of photons emitted at a given instant by the electroluminescent device towards the medium to be analysed (i.e., transmitted via the free zone of the output surface) corresponds to “n−1” times the number of photons detected by the first photodetector at the given instant.

According to one feature of the invention:
the area of the output surface ranges between 6,400 μm² and 14,400 μm², and preferably ranges between 8,100 μm² and 12,100 μm²;
“n” is equal to 2.

Thus, one resulting advantage of this geometric configuration is that it dispenses with an electronic arithmetic computation component, while maintaining satisfactory light distribution between the free zone (and therefore the medium to be analysed) and the first photodetector. Indeed, the number of photons emitted by the electroluminescent device towards the medium to be analysed at a given instant (i.e., transmitted via the free zone of the output surface) then corresponds to the number of photons detected by the first photodetector at the given instant.

According to one feature of the invention, the substrate is made of a material that is reflective in the wavelength range.

Thus, one resulting advantage involves removing a constraint on the first electrode of the electroluminescent device in order to reduce optical losses. Indeed, when the substrate is made of a material that is reflective in the wavelength range, the first electrode of the electroluminescent device may not be reflective in the wavelength range.

Finally, an aim of the invention is a method for manufacturing a diffuse reflectance spectroscopic measurement system for a medium to be analysed, comprising the steps of:
a) using a substrate, comprising a surface intended to be oriented towards the medium to be analysed, the surface having first and second adjacent zones;
b) forming an electroluminescent device on the first zone of the surface of the substrate, the electroluminescent device being configured to emit light radiation in a wavelength range, the electroluminescent device having an output surface, through which the light radiation is transmitted towards the medium to be analysed;
c) forming a first photodetector, called reference photodetector, on a portion of the output surface of the electroluminescent device so as to receive a portion of the light radiation and such that the output surface retains a free zone;
d) forming a second photodetector, called measurement photodetector, on the second zone of the surface of the substrate so as to receive the light radiation transmitted by the free zone of the output surface of the electroluminescent device and diffusely reflected by the medium to be analysed.

Thus, as stated above, such a method according to the invention dispenses with the presence of waveguides compared to the prior art. Indeed, on the one hand, there is no output coupler since the light radiation is transmitted directly via the output surface of the electroluminescent device. On the other hand, the reference photodetector (used to deduce the light power emitted by the electroluminescent device) is arranged on a portion of the output surface of the electroluminescent device. Furthermore, stacking the reference photodetector on the electroluminescent device results in reduced lateral bulk compared to the prior art.

The method according to the invention can comprise one or more of the following features.

According to one feature of the invention, steps c) and d) are concomitant.

Thus, one resulting advantage involves reducing the execution time of the method.

According to one feature of the invention, step b) comprises the steps of:
b₁) forming a first electrode on the first zone of the surface of the substrate;
b₂) forming a stack of layers on the first electrode, the stack comprising an emissive layer configured to emit the light radiation in the wavelength range;
b₃) forming a second electrode, transparent in the wavelength range, on the stack of layers, and having a surface defining the output surface of the electroluminescent device;
in which method the concomitant steps c) and d) comprise the steps of:
   forming a first electrode on a portion of the output surface of the electroluminescent device, as well as on the second zone of the surface of the substrate;
   forming a stack of layers, comprising an absorbent layer configured to absorb the light radiation in the wavelength range, on the first electrode of the first photodetector, as well as on the first electrode of the second photodetector;
   forming a second electrode on the stack of layers of the first photodetector, as well as on the stack of layers of the second photodetector.

According to one feature of the invention, step b) is preceded by the following steps:
forming first contact pads, arranged on the first zone of the surface of the substrate for electrically contacting the second electrode of the electroluminescent device;
forming second contact pads, arranged on the first zone of the surface of the substrate for electrically contacting the second electrode of the first photodetector;
forming third contact pads, arranged on the second zone of the surface of the substrate for electrically contacting the second electrode of the second photodetector;
   in which method:
the second electrode of the electroluminescent device formed during step b₃) extends over the first zone of the surface of the substrate so as to be electrically connected to the first contact pads;
the second electrode of the first photodetector and the second electrode of the second photodetector formed during concomitant steps c) and d) respectively extend over the first and second zones of the surface of the substrate, so as to be respectively electrically connected to the second and third contact pads.

Thus, one resulting advantage involves facilitating electrical connection while limiting the execution time of the method.

### Definitions

“Substrate” is understood to mean a self-supporting physical support made of a base material, from which an electroluminescent device and a photodetector can be formed. A substrate can be a “slice” (also known as a “wafer”) that generally assumes the form of a disc cut from an ingot of crystalline material.
“Wavelength range” is understood to mean a range of values that the wavelength of the light radiation emitted by the electroluminescent device lies within. The range of values can tend towards a single value when the light radiation is monochromatic.
“Free zone” is understood to mean a zone of the output surface of the electroluminescent device that is not covered by the first photodetector (i.e., the reference photodetector).
“Reflective” is understood to mean that the element has an intensity reflection coefficient that is greater than or equal to 60%, preferably greater than or equal to 80%, more preferably greater than or equal to 90%, averaged over the wavelength range.
“Transparent” is understood to mean that the element has an intensity transmission coefficient that is greater than or equal to 60%, preferably greater than or equal to 80%, more preferably greater than or equal to 90%, averaged over the wavelength range.

### BRIEF DESCRIPTION OF THE DRAWINGS

Further features and advantages will become apparent from the detailed disclosure of various embodiments of the invention, which is accompanied by examples and references to the accompanying drawings.

FIG. 1 is a schematic cross-sectional view illustrating a substrate on which an electroluminescent device and a first electrode of the measurement photodetector are formed.

FIG. 2 is a schematic cross-sectional view illustrating the formation of a first electrode of the reference photodetector on a portion of the output surface of the electroluminescent device.

FIG. 3 is a schematic cross-sectional view illustrating the formation of an electrically insulating resin.

FIG. 4 is a schematic cross-sectional view illustrating the formation of stacks of layers, each comprising an absorbent layer, in order to form the reference photodetector and the measurement photodetector.

FIG. 5 is a schematic cross-sectional view illustrating the formation of a second electrode of the reference photodetector and of the measurement photodetector.

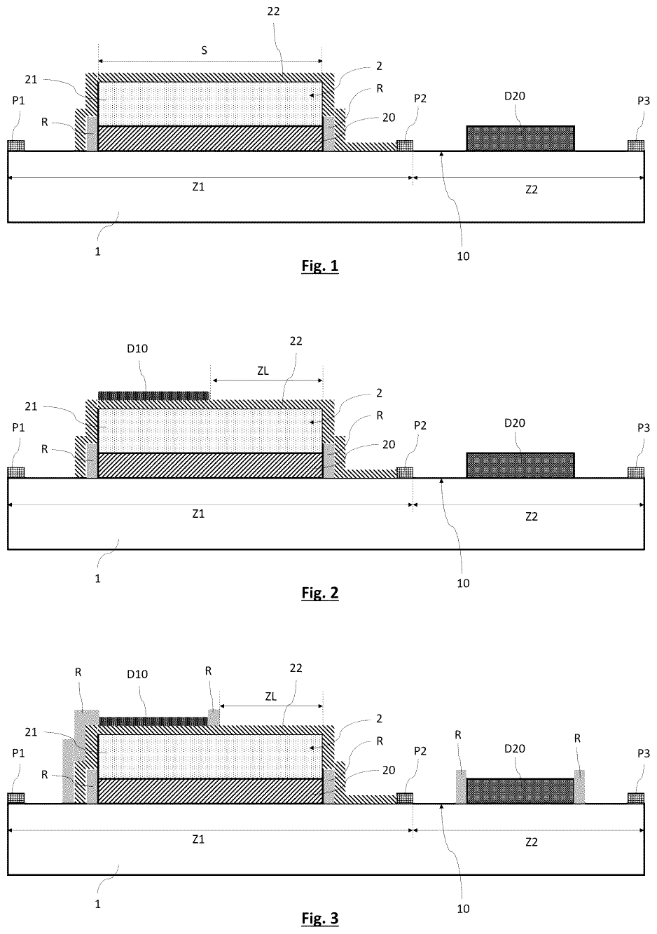

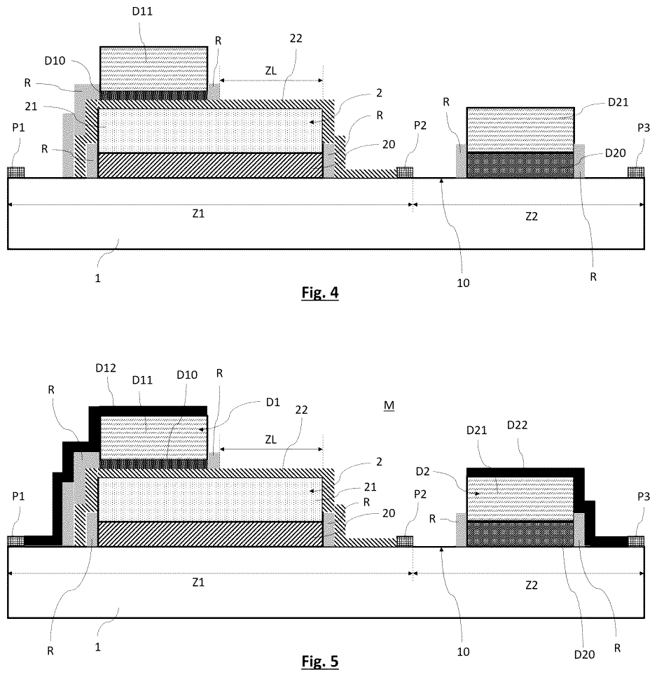

It should be noted that the drawings described above are schematic and are not necessarily to scale for the sake of legibility and for ease of understanding. The cross-sections are provided perpendicular to the surface of the substrate intended to oriented towards the medium to be analysed.

### DETAILED DISCLOSURE OF THE EMBODIMENTS

Identical elements or elements performing the same function will use the same references for the various embodiments, for the sake of simplicity.

### Measurement System

One aim of the invention is a diffuse reflectance spectroscopic measurement system for a medium M to be analysed, comprising:
a substrate 1, comprising a surface 10 intended to be oriented towards the medium M to be analysed, the surface 10 having first and second adjacent zones Z1, Z2;
an electroluminescent device 2, arranged on the first zone Z1 of the surface 10 of the substrate 1, and configured to emit light radiation in a wavelength range, the electroluminescent device 2 having an output surface S, through which the light radiation is transmitted towards the medium M to be analysed;
a first photodetector D1, called reference photodetector, arranged on a portion of the output surface S of the electroluminescent device 2 so as to receive a portion of the light radiation and so that the output surface S retains a free zone ZL;
a second photodetector D2, called measurement photodetector, arranged on the second zone Z2 of the surface 10 of the substrate 1 so as to receive the light radiation transmitted by the free zone ZL of the output surface S of the electroluminescent device 2 and diffusely reflected by the medium M to be analysed.

### Substrate

The substrate 1 comprises a surface 10, preferably a flat surface, intended to be oriented towards the medium M to be analysed. In other words, the surface 10 is intended to face the medium M to be analysed. The surface 10 has first and second adjacent zones Z1, Z2.

According to a first embodiment, the substrate 1 is made of a material that is reflective in the wavelength range. When the wavelength range is the visible domain, the substrate 1 can be made of a material selected from among silicon, a metal coated with an electrical insulator, a flexible polymer-type material (for example, poly(methyl methacrylate), PMMA) coated with a reflective metal layer and an electrical insulator.

According to a second embodiment, the substrate 1 is made of a non-reflective (for example, transparent) material in the wavelength range. When the wavelength range is the visible domain, the substrate 1 can be made of a material selected from among glass or a flexible polymer-type material (for example, poly(methyl methacrylate), PMMA).

By way of a non-limiting example, the thickness of the substrate 1 can range between 50 μm and 1 mm.

### Electroluminescent Device

The electroluminescent device 2 is arranged on the first zone Z1 of the surface 10 of the substrate 1. The electroluminescent device 2 is configured to emit light radiation in a wavelength range. The wavelength range can be the visible domain [380 nm, 850 nm], notably allowing the oxygenation level in skin to be analysed and a pulse wave to be measured. The wavelength range can be the infrared domain, for example, [900 nm, 1600 nm], notably allowing the blood sugar level and the hydration level to be analysed.

The electroluminescent device 2 has an output surface S, through which the emitted light radiation is transmitted towards the medium M to be analysed. By way of a non-limiting example, the output surface S can have an area, denoted “A_{S}”, ranging between 6,400 μm² and 14,400 μm², and preferably ranging between 8,100 μm² and 12,100 μm².

The electroluminescent device 2 advantageously comprises at least one organic light-emitting diode. However, at least one inorganic light-emitting diode can be contemplated for the electroluminescent device 2.

According to one embodiment, the electroluminescent device 2 successively comprises:
a first electrode 20, arranged on the first zone of the surface of the substrate;
a stack 21 of layers, comprising an emissive layer configured to emit the light radiation in the wavelength range;
a second electrode 22, transparent in the wavelength range, and having a surface defining the output surface S of the electroluminescent device 2.

By way of a non-limiting example, the stack 21 of layers can comprise:
a charge injection layer, for example, made of Dipyrazino[2,3-f:2′,3′-h]quinoxaline-2,3,6,7,10,11-hexacarbonitrile;
a charge transport layer, for example, made of 2,2′,7,7′-tetra(N,N-di-p-tolyl)amino-9,9-spirobifluorene;
a charge blocking layer, for example, made of N,N′-Bis(naphthalen-1-yl)-N,N′-bis(phenyl)-benzidine;
an emissive layer, selected according to the wavelength range;
a charge blocking layer, for example, made of N,N′-Bis(3-methylphenyl)-N,N′-bis(phenyl)-benzidine;
a charge transport layer, for example, made of 4,7-Diphenyl-1,10-phenanthroline;
a charge injection layer, for example, made of LiF.

### Reference Photodetector

The first photodetector D1 is arranged on a portion of the output surface S of the electroluminescent device 2 so that the output surface S retains a free zone ZL. The free zone ZL of the output surface S transmits a portion of the light radiation emitted by the electroluminescent device 2 to the medium M to be analysed. The other portion of the light radiation emitted by the electroluminescent device 2 is received by the first photodetector D1.

The first photodetector D1 advantageously comprises at least one organic photodiode.

According to one embodiment, the first photodetector D1 successively comprises:
a first electrode D10, which is transparent in the wavelength range and is arranged on a portion of the output surface S of the electroluminescent device 2;
a stack D11 of layers, comprising an absorbent layer configured to absorb the light radiation in the wavelength range;
a second electrode D12, which is reflective in the wavelength range.

By way of a non-limiting example, the stack D11 of layers can comprise:
a charge transport layer, for example, made of 2,2′,7,7′-Tetra(N,N-di-p-tolyl)amino-9,9-spirobifluorene;
a charge blocking layer, for example, made of N,N′-Bis(naphthalen-1-yl)-N,N′-bis(phenyl)-benzidine;
a photosensitive absorbent layer, selected according to the wavelength range, for example, made of a ZnPc:C60 mixture for the visible domain;
a charge blocking layer, for example, made of N,N′-Bis(3-methylphenyl)-N,N′-bis(phenyl)-benzidine;
a charge transport layer, for example, made of 4,7-Diphenyl-1,10-phenanthroline.

The first electrode D10 of the first photodetector D1 is arranged directly on a portion of the output surface S of the electroluminescent device so that the first electrode D10 of the first photodetector D1 is in contact with the second electrode 22 of the electroluminescent device 2.

The first electrode D10 of the first photodetector D1 is advantageously arranged on a lateral portion of the output surface S of the electroluminescent device 2.

The first electrode D10 of the first photodetector D1 occupies an area, denoted “A_{P}”, advantageously satisfying:

$${A}_{P} = \frac{{A}_{S}}{n}$$

where “n” is a natural number that is greater than or equal to 2.

When the area of the output surface S (“A_{S}”) ranges between 6,400 μm² and 14,400 μm², and preferably ranges between 8,100 μm² and 12,100 μm², then “n” is advantageously equal to 2.

### Measurement Photodetector

The second photodetector D2 is arranged on the second zone Z2 of the surface 10 of the substrate 1 so as to receive the light radiation transmitted by the free zone ZL of the output surface S of the electroluminescent device 2, then diffusely reflected by the medium M to be analysed.

The second photodetector D2 advantageously comprises at least one organic photodiode.

According to one embodiment, the second photodetector D2 successively comprises:
a first electrode D20, which is reflective in the wavelength range and is arranged on the second zone Z2 of the surface 10 of the substrate 1;
a stack D21 of layers, comprising an absorbent layer configured to absorb the light radiation in the wavelength range;
a second electrode D22, transparent in the wavelength range.

More specifically, the absorbent layer of the stack D21 of layers of the second photodetector D2 is configured to absorb the light radiation diffusely reflected by the medium M to be analysed.

By way of a non-limiting example, the stack D21 of layers can comprise:
a charge transport layer, for example, made of 2,2′,7,7′-Tetra(N,N-di-p-tolyl)amino-9,9-spirobifluorene;
a charge blocking layer, for example, made of N,N′-Bis(naphthalen-1-yl)-N,N′-bis(phenyl)-benzidine;
a photosensitive absorbent layer, selected according to the wavelength range, for example, made of a ZnPc:C60 mixture for the visible domain;
a charge blocking layer, for example, made of N,N′-Bis(3-methylphenyl)-N,N′-bis(phenyl)-benzidine;
a charge transport layer, for example, made of 4,7-diphenyl-1,10-phenanthroline.

### Medium to be Analysed

By way of a non-limiting example, the medium M to be analysed is a skin-type biological tissue containing chromophores. In particular, the melanosomes and blood cells of the skin act as centres for diffusing light radiation, dispersed throughout the medium M to be analysed. For example, a person skilled in the art knows how to analyse skin colour (complexion) according to diffuse reflectance measurements based on optical models using Mie theory.

### Encapsulation and Electrical Insulation

Advantageously, the first and second photodetectors D1, D2 are covered with an encapsulation layer designed to protect them from the medium M to be analysed, air, moisture, etc. The encapsulation layer is transparent in the wavelength range. The encapsulation layer can be of the multilayer type. The encapsulation layer is advantageously impermeable to water, water vapour, oxygen, and external aggressions such as sweat, for example. By way of non-limiting examples, the encapsulation layer can comprise at least one material selected from among SiO, TiO₂, Al₂O₃, and parylene.

The system according to the invention advantageously comprises an electrically insulating resin R arranged to insulate:
the first and second electrodes 20, 22 of the electroluminescent device 2 from each other;
the first and second electrodes D10, D12 of the first photodetector D1 from each other;
the first and second electrodes D20, D22 of the second photodetector D2 from each other.

### Manufacturing Method

An aim of the invention is a method for manufacturing a diffuse reflectance spectroscopic measurement system for a medium M to be analysed, comprising the steps of:
a) using a substrate 1, comprising a surface 10 intended to be oriented towards the medium M to be analysed, the surface 10 having first and second adjacent zones Z1, Z2;
b) forming an electroluminescent device 2 on the first zone Z1 of the surface 10 of the substrate 1, the electroluminescent device 2 being configured to emit light radiation in a wavelength range, the electroluminescent device 2 having an output surface S, through which the light radiation is transmitted towards the medium M to be analysed;
c) forming a first photodetector D1, called reference photodetector, on a portion of the output surface S of the electroluminescent device 2 so as to receive a portion of the light radiation and such that the output surface S retains a free zone ZL;
d) forming a second photodetector D2, called measurement photodetector, on the second zone Z2 of the surface 10 of the substrate 1 so as to receive the light radiation transmitted by the free zone ZL of the output surface S of the electroluminescent device 2 and diffusely reflected by the medium M to be analysed.

### Step a)

Step a) involves using a substrate 1, comprising a surface 10 intended to be oriented towards the medium M to be analysed. In other words, the surface 10 is intended to face the medium M to be analysed. The surface 10 has first and second adjacent zones Z1, Z2. The technical features of the substrate 1 described above apply to step a) of the method according to the invention.

### Step b)

Step b) involves forming an electroluminescent device 2 on the first zone Z1 of the surface 10 of the substrate 1. The electroluminescent device 2 is configured to emit light radiation in a wavelength range. The electroluminescent device 2 has an output surface S, through which the light radiation is transmitted towards the medium M to be analysed. The technical features of the electroluminescent device 2 described above apply to step b) of the method according to the invention.

Step b) can comprise the steps of:
b₁) forming a first electrode 20 on the first zone Z1 of the surface 10 of the substrate 1;
b₂) forming a stack 21 of layers on the first electrode 20, the stack 21 comprising an emissive layer configured to emit the light radiation in the wavelength range;
b₃) forming a second electrode 22, transparent in the wavelength range, on the stack 21 of layers, and having a surface defining the output surface S of the electroluminescent device 2.

### Step c)

Step c) involves forming a first photodetector D1, called reference photodetector, on a portion of the output surface S of the electroluminescent device 2 so that the output surface S retains a free zone ZL. The free zone ZL of the output surface S transmits a portion of the light radiation emitted by the electroluminescent device 2 towards the medium M to be analysed. The other portion of the light radiation emitted by the electroluminescent device 2 is received by the first photodetector D1.

The technical features of the first photodetector D1 described above apply to step c) of the method according to the invention.

### Step d)

Step d) involves forming a second photodetector D2, called measurement photodetector, on the second zone Z2 of the surface 10 of the substrate 1. The second photodetector D2 is arranged on the second zone Z2 of the surface 10 of the substrate 1 so as to receive the light radiation transmitted by the free zone ZL of the output surface S of the electroluminescent device 2, then diffusely reflected by the medium M to be analysed.

The technical features of the second photodetector D2 described above apply to step d) of the method according to the invention.

Steps c) and d) are advantageously concomitant. The concomitant steps c) and d) advantageously comprise the steps of:
forming a first electrode D10, D20 on a portion of the output surface S of the electroluminescent device 2, as well as on the second zone Z2 of the surface 10 of the substrate 1;
forming a stack D11, D21 of layers, comprising an absorbent layer configured to absorb light radiation in the wavelength range, on the first electrode D10 of the first photodetector D1, as well as on the first electrode D20 of the second photodetector D2;
forming a second electrode D12, D22 on the stack D11 of layers of the first photodetector D1, as well as on the stack D21 of layers of the second photodetector D2.

### Contact Pads

Step b) is advantageously preceded by steps of:
forming first contact pads P1, arranged on the first zone Z1 of the surface 10 of the substrate 1 for electrically contacting the second electrode 22 of the electroluminescent device 2;
forming second contact pads P2, arranged on the first zone Z1 of the surface 10 of the substrate 1 for electrically contacting the second electrode D12 of the first photodetector D1;
forming third contact pads P3, arranged on the second zone Z2 of the surface 10 of the substrate 1 for electrically contacting the second electrode D22 of the second photodetector D2.

The second electrode 22 of the electroluminescent device 2 formed during step b₃) advantageously extends over the first zone Z1 of the surface 10 of the substrate 1 so as to be electrically connected to the first contact pads P1.

Advantageously, the second electrode D12 of the first photodetector D1 and the second electrode D22 of the second photodetector D2 formed during concomitant steps c) and d) respectively extend over the first and second zones Z1, Z2 of the surface 10 of the substrate 1, so as to be electrically connected to the second and third contact pads P2, P3, respectively.

Alternatively, the second contact pads P2 and the third contact pads P3 can be electrically connected to each other. If applicable, the second electrode D12 of the first photodetector D1 and the second electrode D22 of the second photodetector D2 can be shared.

The invention is not limited to the disclosed embodiments. A person skilled in the art will be able to consider technically effective combinations thereof and substitute them with equivalents.

1. A diffuse reflectance spectroscopic measurement system for a medium to be analysed, comprising:
a substrate, comprising a surface intended to be oriented towards the medium to be analysed, the surface having first and second adjacent zones;
an electroluminescent device, arranged on the first zone of the surface of the substrate, and configured to emit light radiation in a wavelength range, the electroluminescent device having an output surface, through which the light radiation is transmitted towards the medium to be analysed, the output surface having an area, denoted “A_{S}”;
a first photodetector, called reference photodetector, arranged on a portion of the output surface of the electroluminescent device so as to receive a portion of the light radiation and so that the output surface retains a free zone, the first photodetector successively comprises:
a first electrode, transparent in the wavelength range, and arranged on a portion of the output surface of the electroluminescent device, the first electrode of the first photodetector occupies an area, denoted “A_{P}”, satisfying:
$${A}_{P} = \frac{{A}_{S}}{n}$$
where “n” is a natural number that is greater than or equal to 2;
a stack of layers, comprising an absorbent layer configured to absorb the light radiation in the wavelength range;
a second electrode, which is reflective in the wavelength range;
a second photodetector, called measurement photodetector, arranged on the second zone of the surface of the substrate so as to receive the light radiation transmitted by the free zone of the output surface of the electroluminescent device and diffusely reflected by the medium to be analysed.

2. The system according to claim 1, wherein the electroluminescent device comprises at least one organic light-emitting diode.

3. The system according to claim 1, wherein the first photodetector comprises at least one organic photodiode.

4. The system according to claim 1, wherein the second photodetector comprises at least one organic photodiode.

5. The system according to claim 1, wherein the electroluminescent device successively comprises:
a first electrode, arranged on the first zone of the surface of the substrate;
a stack of layers, comprising an emissive layer configured to emit the light radiation in the wavelength range;
a second electrode, transparent in the wavelength range, and having a surface defining the output surface of the electroluminescent device.

6. The system according to claim 1, wherein the first electrode of the first photodetector is arranged on a lateral portion of the output surface.

7. The system according to claim 1, wherein:
the area of the output surface ranges between 6,400 μm² and 14,400 μm², and preferably ranges between 8,100 μm² and 12,100 μm²;
“n” is equal to 2.

8. The system according to claim 1, wherein the substrate is made of a material that is reflective in the wavelength range.

9. A method for manufacturing a diffuse reflectance spectroscopic measurement system for a medium to be analysed, comprising the steps of:
a) using a substrate, comprising a surface intended to be oriented towards the medium to be analysed, the surface having first and second adjacent zones;
b) forming an electroluminescent device on the first zone of the surface of the substrate, the electroluminescent device being configured to emit light radiation in a wavelength range, the electroluminescent device having an output surface, through which the light radiation is transmitted towards the medium to be analysed, the output surface having an area, denoted “A_{S}”;
c) forming a first photodetector, called reference photodetector, on a portion of the output surface of the electroluminescent device so as to receive a portion of the light radiation and such that the output surface retains a free zone, the first photodetector successively comprises:
a first electrode, transparent in the wavelength range, and arranged on a portion of the output surface of the electroluminescent device, the first electrode of the first photodetector occupies an area, denoted “A_{P}”, satisfying:
$${A}_{P} = \frac{{A}_{S}}{n}$$
where “n” is a natural number that is greater than or equal to 2;
a stack of layers, comprising an absorbent layer configured to absorb the light radiation in the wavelength range;
a second electrode, which is reflective in the wavelength range;
d) forming a second photodetector, called measurement photodetector, on the second zone of the surface of the substrate so as to receive the light radiation transmitted by the free zone of the output surface of the electroluminescent device and diffusely reflected by the medium to be analysed.

10. The method according to claim 9, wherein steps c) and d) are concomitant.

11. The method according to claim 10, wherein step b) comprises the steps of:
b₁) forming a first electrode on the first zone of the surface of the substrate;
b₂) forming a stack of layers on the first electrode, the stack comprising an emissive layer configured to emit light radiation in the wavelength range;
b₃) forming a second electrode, transparent in the wavelength range, on the stack of layers, and having a surface defining the output surface of the electroluminescent device;
in which method the concomitant steps c) and d) comprise the steps of:
forming a first electrode on a portion of the output surface of the electroluminescent device, as well as on the second zone of the surface of the substrate;
forming a stack of layers, comprising an absorbent layer configured to absorb light radiation in the wavelength range, on the first electrode of the first photodetector, as well as on the first electrode of the second photodetector;
forming a second electrode on the stack of layers of the first photodetector, as well as on the stack of layers of the second photodetector.

12. The method according to claim 11, wherein step b) is preceded by steps of:
forming first contact pads, arranged on the first zone of the surface of the substrate for electrically contacting the second electrode of the electroluminescent device;
forming second contact pads, arranged on the first zone of the surface of the substrate for electrically contacting the second electrode of the first photodetector;
forming third contact pads, arranged on the second zone of the surface of the substrate for electrically contacting the second electrode of the second photodetector;
in which method:
the second electrode of the electroluminescent device formed during step b₃) extends over the first zone of the surface of the substrate so as to be electrically connected to the first contact pads;
the second electrode of the first photodetector and the second electrode of the second photodetector formed during concomitant steps c) and d) respectively extend over the first and second zones of the surface of the substrate, so as to be respectively electrically connected to the second and third contact pads.
